(19) United States
(12) Patent Application Publication
(10) Pub. No.: US 2015/0160310 A1
(43) Pub. Date: Jun. 11, 2015

(54) RADIO-FREQUENCY COIL SYSTEM FOR AN INTERVENTIONAL MAGNETIC RESONANCE EXAMINATION

(71) Applicant: Siemens Aktiengesellschaft, Muenchen (DE)

(72) Inventors: Martin Requardt, Nuernberg (DE); Martin Ringholz, Erlangen (DE)

(73) Assignee: SIEMENS AKTIENGESELLSCHAFT, Muenchen (DE)

(21) Appl. No.: 14/561,495

(22) Filed: Dec. 5, 2014

(30) Foreign Application Priority Data
Dec. 5, 2013 (DE) 102013224981.0

(51) Int. Cl. G01R 33/28; A61B 19/00; A61N 5/10; G01R 33/34

(52) U.S. Cl. CPC G01R 33/286; G01R 33/34; A61B 19/201; A61N 5/1001

(57) ABSTRACT

A coil system for an interventional magnetic resonance examination has at least one penetration template, and at least one radio-frequency coil that has at least one opening therein, with the shape of the at least one opening is matched to the shape of the at least one penetration template so that the at least one penetration template can be integrated into the at least one opening.

### BACKGROUND OF THE INVENTION

1. Field of the Invention

The invention concerns: a radio-frequency (RF) coil system for an interventional magnetic resonance examination, a radio-frequency coil, and a penetration template.

2. Description of the Prior Art

For conventional interventional magnetic resonance examinations, radio-frequency (RF) coils for magnetic resonance imaging are typically used separately from penetration templates that, for example, are suitable for guidance of interventional devices for puncturing or for introduction of radiation sources for brachytherapy.

### SUMMARY OF THE INVENTION

An object of the invention is to enable an improved interventional magnetic resonance examination.

A coil system in accordance with the invention for an interventional magnetic resonance has at least one penetration template and at least one radio-frequency coil, the at least one radio-frequency coil having at least one opening therein, with the shape of the at least one opening matched to the shape of the at least one penetration template such that the at least one penetration template can be integrated into the at least one opening.

The at least one radio-frequency coil typically is formed by one or more radio-frequency antennas and is used to acquire radio-frequency signals (also called magnetic resonance signals) during magnetic resonance examinations. The at least one radio-frequency coil is thus a component of a magnetic resonance apparatus. The at least one radio-frequency coil is typically used in order to improve the signal quality (in particular the signal-to-noise ratio) of the received magnetic resonance signals due to being able to be placed in close spatial proximity to the examination region. The at least one radio-frequency coil is preferably fashioned as a local radio-frequency coil (in particular a surface coil) and/or as a flexibly designed radio-frequency coil. For the interventional magnetic resonance examination in particular, the at least one radio-frequency coil is arranged on a patient, specifically at an examination region of the patient and/or at a body region of the patient that is designated for an interventional procedure. The at least one radio-frequency coil can then also be adapted to the anatomical conditions of the patient with regard to the body region designated for the interventional procedure. For example, for penetration of the prostate of the patient, the at least one radio-frequency coil is advantageously shaped so as to be placeable at the perineum region of the patient, so the at least one radio-frequency coil can include cutouts for the legs of the patient. For a spinal penetration (tap), the at least one radio-frequency coil is advantageously shaped so as to ne placeable in the back region of the patient.

The at least one penetration template—possibly a biopsy penetration template—typically includes one or more guide devices (for example guide holes) that provide a trajectory for an interventional device, for example a penetration needle and/or a catheter. If the at least one penetration template includes multiple guide devices, these are typically arranged in the form of a grid and, for example, can be provided with coordinates. The penetration template is then typically also called a grid or biopsy grid. The at least one penetration template is thereby advantageously positioned and/or designed such that it ensures that a previously assigned body region that is designated for the interventional procedure can be reached with the interventional device. The body region that is designated for the interventional procedure is thereby advantageously designated and/or limited by means of magnetic resonance imaging, in particular, using the at least one radio-frequency coil.

In conventional interventional magnetic resonance examinations, the radio-frequency coil and penetration template are typically not matched to one another. In conventional interventional magnetic resonance examinations, the penetration template is arranged in large openings and/or in the boundary region of the radio-frequency coil. The quality of the magnetic resonance signals received by the radio-frequency coil thus can be degraded. Access to the penetration template—in particular for the interventional device—by the radio-frequency coil also may be hindered.

In contrast to this, the coil system according to the invention enables an advantageous matching of the at least one radio-frequency coil to the at least one penetration template. The coil system according to the invention can also include multiple radio-frequency coils and/or penetration templates, in particular with different characteristics for different applications. The at least one radio-frequency coil advantageously has at least one opening matched to the at least one penetration template. The at least one opening is designed to acquire the at least one penetration template. The at least one opening is thereby advantageously arranged in a central region of the at least one radio-frequency coil since an advantageous signal reception characteristic of the at least one radio-frequency coil is typically present there. The at least one penetration template and/or the at least one radio-frequency coil is then advantageously arranged such that an improved interventional magnetic resonance imaging is possible. For an interventional procedure and/or an interventional examination, the at least one penetration template can particularly simply be inserted into the at least one opening and/or be removed from the at least one opening. The workflow during an interventional procedure and/or interventional examination can therefore be markedly improved. The coil system thus facilitates the interventional procedure and simultaneously ensures an optimal image quality of the magnetic resonance images which are reconstructed using magnetic resonance signals received by means of the at least one radio-frequency coil. The coil system thus contributes to the safety in an interventional procedure. The coil system is advantageously designed so as to ensure sterile conditions during the interventional procedure.

A combined application of the at least one penetration template and of the at least one radio-frequency coil is therefore useful since the at least one radio-frequency coil can enable a fast magnetic resonance imaging, which enables tracking (in particular time-resolved tracking, possibly in real time) of an interventional device within the body of the patient, which interventional device is inserted into a guide device of the at least one penetration template. The guide device typically appears as a signal cancelation in the magnetic resonance images. The penetration of the interventional device into the body of the patient (in particular up to the target region) thus can be tracked in the interventional magnetic resonance images. A prior depth determination can thus also be omitted.

In an embodiment, the at least one penetration template can be integrated with a positive fit into the at least one opening. The at least one opening thus has a shape that corresponds to the shape of the at least one penetration template. “Positive fit” means that at least one contact point and/or an at least a partial, but preferably complete, contact surface exists between the inner wall of the at least one opening and the adjoining outer wall of the at least one penetration template. Preferably, no play then exists between the inner wall and the adjoining outer wall. The material of the at least one radio-frequency coil that surrounds the at least one opening is thereby advantageously fashioned so as to be deformable, for example from plastic and/or foamed material. The at least one penetration template can then be capable of integration under pressure into the at least one opening, and be fixed positively in the deformable material of the at least one radio-frequency coil. The positive integration of the at least one penetration template in the at least one opening leads to a particularly secure and solid retention of the at least one penetration template in the at least one opening, and enables a particularly simple placement and/or fixing of the at least one penetration template in the at least one opening, such as by the application of pressure.

In another embodiment the at least one radio-frequency coil has at least one first mount which is arranged at the at least one opening and is matched to at least one first counterpart of the at least one penetration template such that the at least one penetration template can be fixed within the at least one opening by the at least one first mount. The at least one first mount and/or the at least one first counterpart can thereby comprise at least one attachment element, for example a plug connection and/or hook-and-loop connection and/or adhesive connection and/or screw connection. The at least one first mount of the at least one radio-frequency coil is advantageously a retention frame surrounding the at least one opening. The retention frame is advantageously fashioned from a deformable material, for example from foamed material. The at least one first counterpart of the at least one penetration template is then advantageously a guide lug and/or a protuberance. Upon pressing the at least one penetration template into the at least one opening, the retention frame then adapts to the shape of the at least one first counterpart of the at least one penetration template, such that the at least one penetration template is firmly fixed in the at least one opening. The retention frame preferably has another guide channel hat is fashioned to accommodate the guide lug and/or the protuberance of the at least one penetration template. The at least one penetration template can thus be securely fixed in the at least one opening, and can also be removed from this again particularly simply.

In another embodiment, the at least one penetration template has at least one second mount which is matched to at least one second counterpart of the at least one radio-frequency coil such that the at least one penetration template can be fixed within the at least one opening by the at least one second mount. In particular, the techniques described in the previous paragraph apply to this embodiment. In particular, the at least one opening of the at least one radio-frequency coil can have a retention frame which comprises a guide lug. Such a guide lug of the retention frame is fashioned as a second counterpart to a guide channel of the at least one penetration template. The guide channel of the at least one penetration template is then in particular fashioned as a second mount. It is likewise advantageous for each of the at least one radio-frequency coil and the at least one penetration template to have at least one mount and at least one counterpart. A particularly secure retention of the at least one penetration template in the at least one opening can therefore be ensured.

In another embodiment the at least one penetration template can be affixed reversibly (removably) within the at least one opening. For this, the at least one first mount and the at least one first counterpart and/or the at least one second mount and the at least one second counterpart can be suitably designed so that the at least one penetration template can be reversibly affixed within the at least one opening. After a fixing of the at least one penetration template in the at least one opening, the at least one penetration template can thus be removed again from the at least one opening, in particular without residues and/or additional tools. A particularly versatile usage capability for the at least one coil system is thus achieved since, for example, the at least one radio-frequency coil can be used for specific applications, even without the at least one penetration template.

In another embodiment the at least one penetration template is designed to be magnetic resonance-compatible, such as by being formed partially, or preferably nearly entirely, of non-magnetic materials (such as plastic). By the use of non-magnetic materials, influencing of the magnetic fields or radio-frequency waves in the magnetic resonance apparatus during the use of the at least one penetration template is then typically reduced or precluded. The use of non-magnetic materials also typically prevents artifacts from arising in the images acquired by the magnetic resonance apparatus. The at least one radio-frequency coil is typically also designed to be magnetic resonance-compatible, such that the entire coil system is then advantageously designed to be magnetic resonance-compatible.

In another embodiment the coil system has at least one magnetic resonance marking element which is arranged at the at least one opening and/or the at least one penetration template. The at least one magnetic resonance marking element is magnetic resonance-visible. This means that the at least one magnetic resonance marking element has a contrast relative to surrounding structures and/or can be demarcated from surrounding structures in magnetic resonance images that are acquired with a field of view which includes the at least one magnetic resonance marking element. Hardware components (for example the at least one radio-frequency coil and/or the at least one penetration template) are typically not magnetic resonance-visible, and have no contrast (relative to air, for example) in magnetic resonance images. The at least one magnetic resonance marking element therefore leads to the situation that the position of the at least one opening and/or of the at least one penetration template is visible in magnetic resonance images. The at least one magnetic resonance marking element can thus lead to a visualization of the at least one opening and/or of the at least one penetration template in the magnetic resonance images. The at least one magnetic resonance marking element—in particular the image information of the at least one magnetic resonance marking element during an interventional magnetic resonance examination—serves as an orientation aid during an interventional procedure. The magnetic resonance marking element can thus offer a starting point in magnetic resonance images which, for example, facilitates and/or enables a selection of a matching guide hole for an interventional device. The at least one magnetic resonance marking element therefore advantageously enables a virtual depiction of the at least one penetration template (in particular of the at least one guide hole of the at least one penetration template) in the magnetic resonance images. A virtual depth determination in the magnetic resonance images is then also possible for the interventional device at the target region. The magnetic resonance images can thus include a virtual (in particular three-dimensional) representation of target markers for the interventional device with regard to the target region in the body of the patient. This is possible in a simple manner when the at least one guide hole of the at least one penetration template is fashioned orthogonal to the at least one penetration template and/or the at least one opening. The at least one magnetic resonance marking element is advantageously arranged at the at least one opening and duplicates (conforms to) the shape of the at least one opening. Alternatively or additionally, the at least one magnetic resonance marking element can be attached to the at least one penetration template, so the marking of the position of the at least one penetration template becomes more precise, but handling of the at least one penetration template may be hindered. An arrangement of the at least one magnetic resonance marking element in a boundary region of the at least one surface coil provides a particularly simple handling, but the precision of the marking of the position of the at least one opening is then limited.

In another embodiment, the at least one magnetic resonance marking element comprises at least one channel which is designed to accommodate a magnetic resonance-visible fluid. The at least one channel can be arranged on the inner wall of the at least one opening and/or surround the at least one opening, in particular along its periphery. Alternatively or additionally, the at least one channel can surround the at least one penetration template at its outer wall, in particular along the periphery of the at least one penetration template. Alternatively or additionally, the at least one channel can be integrated into the inner wall of the at least one opening and/or into the outer wall of the at least one penetration template. The at least one channel advantageously has an intake opening (in particular an intake valve) into which the fluid can be introduced into the at least one channel. The at least one channel can likewise have an outlet opening (in particular an outlet valve) to let out the fluid. The fluid can then be injected into the at least one channel via the intake opening, for example by means of an injector and/or a syringe. The at least one channel can thus be filled with fluid as needed. The at least one channel can also be flexibly adapted in its shape to the at least one opening and/or the at least one penetration template. The at least one channel thus forms an advantageous and versatile magnetic resonance marking element.

In another embodiment, the magnetic resonance-visible fluid is a magnetic resonance contrast agent. Magnetic resonance contrast agents are normally designed in order to cause particularly high or particularly low magnetic resonance signals. The at least one channel filled with the magnetic resonance contrast agent is thus clearly emphasized in the magnetic resonance images, which enables a clear marking of the position of the at least one opening and/or the at least one penetration template.

In another embodiment, the coil system has a plan (planning) template, wherein the plan template can be integrated into the at least one opening and has a magnetic resonance-visible coordinate system. The shape of the plan template is advantageously matched to the shape of the at least one opening such that the plan template can be integrated—in particular with positive fit—into the at least one opening. The shape of the plan template corresponds to the shape of the penetration template. Like the penetration template, the plan template can be fixed (in particular reversibly) within the at least one opening by at least one mount. The magnetic resonance-visible coordinate system of the plan template thereby advantageously enables reading of the coordinates of the coordinate system in magnetic resonance images acquired by the at least one radio-frequency coil if the plan template is integrated into the at least one opening. The plan template thus advantageously enables a virtual representation of the coordinates within the magnetic resonance images. The plan template thus enables a determination of the coordinates in the coordinate system for the interventional procedure, in particular using the acquired magnetic resonance images. After the determination of the coordinates, the plan template can be exchanged for the at least one penetration template, for which the in particular reversible fixing of the plan template and/or of the at least one penetration template is advantageous. The at least one penetration template can have an additional coordinate system, in particular printed on the outside of the at least one penetration template. The additional coordinate system of the at least one penetration template preferably corresponds to the magnetic resonance-visible coordinate system of the plan template. The additional coordinate system of the at least one penetration template is thereby advantageously designed similar (in particular identical) to the coordinate system of the plan template. During the interventional procedure, guidance of the interventional device along the at least one interventional guide device of the at least one penetration template can then take place using the additional coordinate system of the at least one penetration template and the planning using the magnetic resonance-visible coordinate system of the plan template. The plan template with the magnetic resonance-visible coordinate system enables a particularly effective and intuitive planning of the interventional procedure using the magnetic resonance images.

The radio-frequency coil according to the invention for a magnetic resonance apparatus has at least one opening, wherein the shape of the at least one opening is matched to the shape of a penetration template such that the penetration template can be integrated into the at least one opening.

In an embodiment, the radio-frequency coil has at least one first mount which is arranged at the at least one opening and is matched to at least a first counterpart of the penetration template such that said penetration template can be fixed within the at least one opening by the at least first mount. Additional embodiments of the radio-frequency coil according to the invention are fashioned analogous to the embodiments of the at least one radio-frequency coil of the coil system according to the invention. The advantages of the radio-frequency coil according to the invention essentially correspond to the advantages of the coil system according to the invention that are described above, in particular with regard to the at least one radio-frequency coil of the coil system.

The penetration template according to the invention for an interventional magnetic resonance examination has at least one guide device for an interventional device and at least one second mount that is matched to at least one second counterpart of a radio-frequency coil such that the penetration template can be fixed within an opening of the radio-frequency coil. Additional embodiments of the penetration template according to the invention are fashioned analogous to the embodiments of the at least one penetration template of the coil system according to the invention. The advantages of the penetration template according to the invention essentially correspond to the advantages of the coil system according to the invention that are described above, in particular with regard to the at least one penetration template of the coil system.

### BRIEF DESCRIPTION OF THE DRAWINGS

FIG. 1 shows a first embodiment of a radio-frequency coil according to the invention.

FIG. 2 shows a first embodiment of a penetration template according to the invention.

FIG. 3 shows a first embodiment of a coil system according to the invention.

FIG. 4 shows a second embodiment of a radio-frequency coil according to the invention.

FIG. 5 shows a second embodiment of a penetration template according to the invention.

FIG. 6 shows a second embodiment of a coil system according to the invention.

### DESCRIPTION OF THE PREFERRED EMBODIMENTS

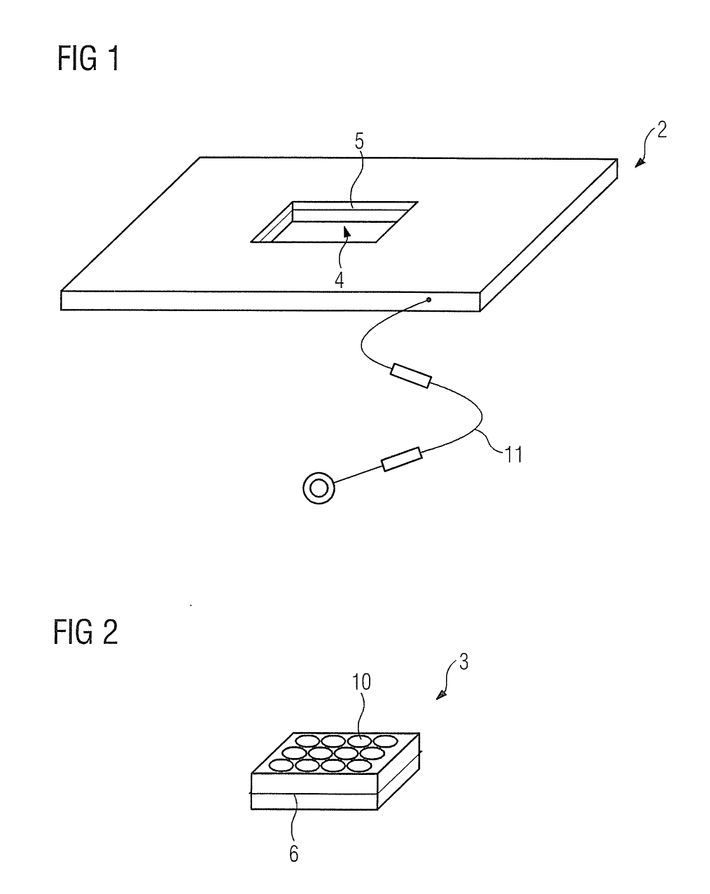

FIG. 1 shows a first embodiment of a radio-frequency coil 2 according to the invention in a schematic illustration. The radio-frequency coil has a connecting element 11 that, in the illustrated embodiment, is fashioned as a cable. The connecting element 11 enables a connection (in particular a data transfer) from the radio-frequency coil 2 to a magnetic resonance apparatus (not shown). In particular, the radio-frequency coil 2 serves to receive magnetic resonance signals, in particular from an examination subject positioned in the magnetic resonance apparatus.

The radio-frequency coil 2 furthermore has an opening 4, wherein the shape of the opening 4 is matched to the shape of the penetration template 3 (see FIG. 2 or FIG. 5) such that the penetration template 3 can be integrated into the opening 4. In the shown case, the opening 4 is arranged in the central region of the radio-frequency coil 2 since a good signal reception characteristic of the radio-frequency coil 2 is present there.

Furthermore, the radio-frequency coil 2 has a first mount 5 which is arranged at the opening 4. The first mount 5 is matched to a first counterpart 6 of the penetration template 3 (see FIG. 2) such that the penetration template 3 can be fixed (in particular can be reversibly fixed) within the opening 4 by means of the first mount 5. In the shown case, the first mount 5 is fashioned as a guide frame made of foamed material. The guide frame has a recess—a first guide channel 5—which is arranged along the entire inner circumference of the opening 4.

FIG. 2 shows a first embodiment of a penetration template 3 according to the invention in a schematic illustration. The penetration template 3 includes multiple guide devices 10 (in the shown case, guide holes 10) which provide trajectories for an interventional device, for example a penetration needle and/or a catheter. The guide holes 10 are arranged in the form of a grid and designed orthogonal to the penetration template 3.

Furthermore, the shape of the penetration template 3 is matched to the shape of the opening 4 of the radio-frequency coil 2 (see FIG. 1) such that the penetration template 3 can be integrated into the opening 4.

Furthermore, the penetration template 3 has a first counterpart 6 which is matched to the first mount 5 of the radio-frequency coil 2 such that the penetration template 3 can be fixed within the opening 4 by the first mount 5, in particular can be reversibly fixed. In the shown case, the first counterpart 6 is fashioned as a projection, in particular as a first guide lug 6. The first guide lug 6 thereby surrounds the full outer circumference of the penetration template 3 and is matched in terms of its shape and size to the shape and size of the first mount 5 of the radio-frequency coil 2, the first guide channel 5.

The penetration template 3 is designed to be magnetic resonance-compatible. The penetration template 3 is therefore composed entirely of non-magnetic materials, in particular plastic.

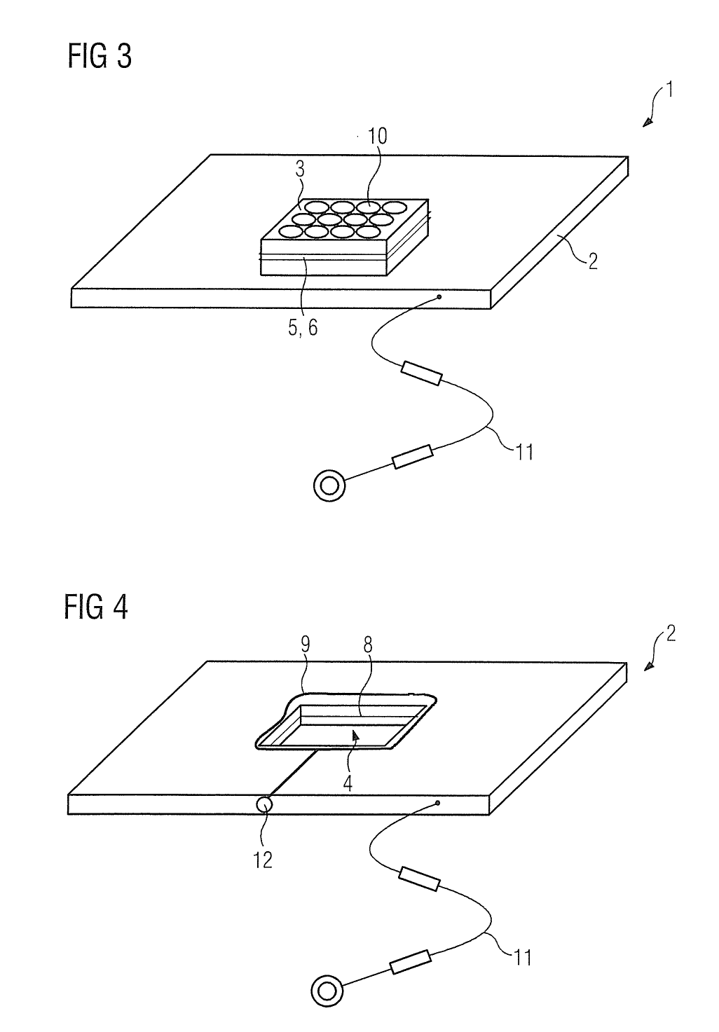

FIG. 3 shows a first embodiment of a coil system 1 according to the invention in a schematic presentation. The coil system 1 includes the radio-frequency coil 2 shown in FIG. 1 and the penetration template 3 shown in FIG. 2.

The penetration template 3 has been integrated into the opening 4 of the radio-frequency coil 2 by pressing. Upon pressing the penetration template 3 into the opening 4, the first mount 5 of the radio-frequency coil 2 (the retention frame with the first guide channel 5) is adapted to the shape of the first counterpart 6 of the penetration template 3 (the guide lug 6) so that the penetration template 3 is firmly fixed in the opening 4, but can also be simply removed from the opening 4 again.

From FIG. 3 it can be seen that the shape of the opening 4 is matched to the shape of the penetration template 3 such that the penetration template 3 is integrated with positive fit into the opening 4. The inner wall of the opening 4 thereby rests with its entire circumference on the outer wall of the penetration template 3, in particular the side wall of the penetration template 3. There is thus no free space between the inner wall of the opening 4 and the penetration template 3, such that the penetration template 3 is fixed particularly securely within the opening 4.

FIG. 4 shows a second embodiment of a radio-frequency coil 2 according to the invention in a schematic illustration. The radio-frequency coil 2 shown in FIG. 4 has essentially the same elements as the radio-frequency coil 2 shown in FIG. 1.

However, the radio-frequency coil 2 according to FIG. 4 additionally has a magnetic resonance marking element 9 arranged at the opening 4. The magnetic resonance marking element 9 is fashioned as a channel 9 which is arranged around the opening 4, along the circumference of said opening 4. The channel 9 is designed to receive a magnetic resonance-visible fluid, in particular a magnetic resonance contrast agent. For this, the channel 9 has an intake opening 12 (in particular an intake valve 12) into which the fluid can be introduced. The fluid can also be let out from the channel 9 again as needed via an outlet opening (not shown), in particular an output valve. Insofar as it is filled with the magnetic resonance-visible fluid, the magnetic resonance marking element 9 can serve during an interventional magnetic resonance examination as an orientation aid to show the position of the opening 4, and thus the position of the penetration template 3 fixed in the opening 4.

Furthermore, instead of the first mount 5 the radio-frequency coil 2 according to FIG. 4 has a second counterpart 8 to which a second mount 7 of the penetration template 3 (see FIG. 5) is matched, such that the penetration template 3 can be fixed within the opening 4 by means of the second mount 7. In the illustrated embodiment, the second counterpart is fashioned as a second guide lug 8. Naturally, the radio-frequency coil 2 from FIG. 4 can also have a first mount 5, a first guide channel 5.

FIG. 5 shows a second embodiment of a penetration template 3 according to the invention in a schematic illustration. The penetration template 3 shown in FIG. 5 has essentially the same elements as the penetration template 3 shown in FIG. 2.

However, the penetration template 3 according to FIG. 5 additionally has a magnetic resonance marking element 9 which is arranged on the penetration template 3, in particular the outer wall of the penetration template 3. As previously described, the magnetic resonance marking element 9 is fashioned as a channel 9 with an intake opening 12.

Furthermore, instead of the first counterpart 6 the penetration template 3 according to FIG. 5 has a second mount 7 to which the second counterpart 8 of the radio-frequency coil 2 (see FIG. 5) is matched, such that the penetration template 3 can be fixed within the opening 4 by means of the second mount 7. In the illustrated embodiment, the second mount 7 is fashioned as a second guide channel 7. The second guide channel 7 is matched in terms of its shape and size to the shape and size of the second counterpart 8 of the radio-frequency coil 2 (the second guide lug 8). Naturally, the penetration template 3 from FIG. 5 can also have a first counterpart 6 (a first guide lug 6).

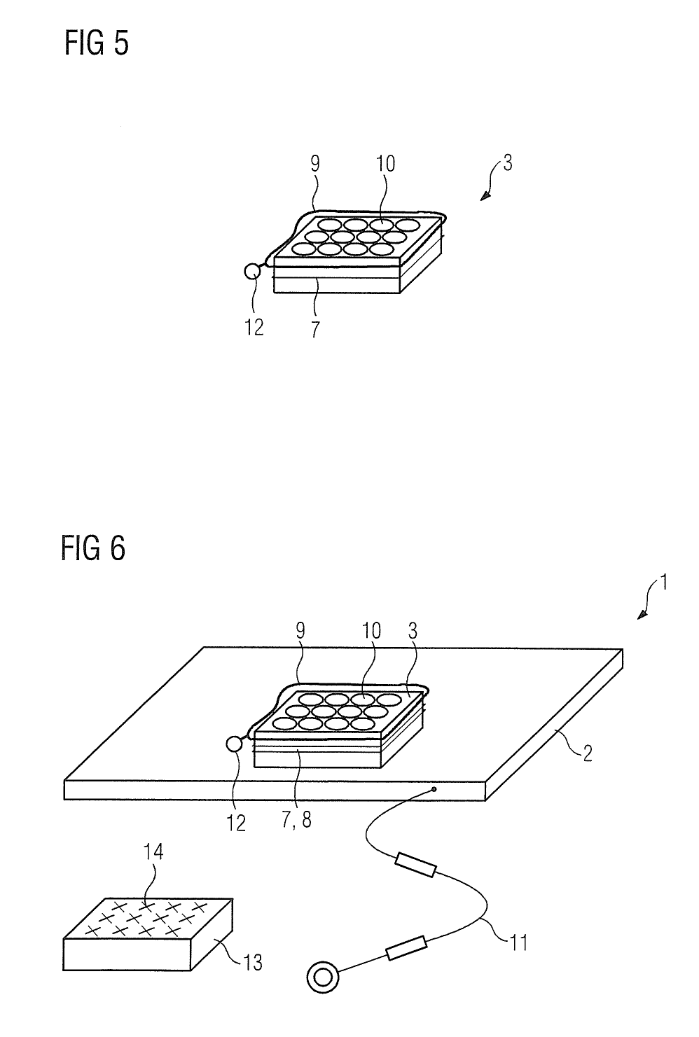

FIG. 6 shows a second embodiment of a coil system 1 according to the invention in a schematic illustration. The coil system 1 has the radio-frequency coil 2 shown in FIG. 4 and the penetration template 3 shown in FIG. 5.

The position of the penetration template 3 fixed within the opening 4 of the radio-frequency coil 2 is thus indicated by the channel 9 which is arranged at the opening 4. The channel 9 of the penetration template 3 was removed before the integration of the penetration template 3 into the opening 4, since a channel 9 for marking the position of the penetration template 3 is sufficient. Although not shown, the coil system 1 can have two channels 9: the channel 9 of the radio-frequency coil 2 according to FIG. 4 and the channel 9 of the penetration template 3 according to FIG. 5. A more precise marking of the position of the penetration template 3 would therefore be possible.

The penetration template 3 is again fixed within the opening 4 via the second mount 7 of the penetration template 3 and the second counterpart 8 of the radio-frequency coil 2.

Furthermore, the coil system 1 according to the invention has a plan template 13, wherein the plan template 13 can be integrated into the at least one opening 4 and has a magnetic resonance-visible coordinate system 14. The plan template 13—in particular the magnetic resonance-visible coordinate system 14—enables a coordinate determination in magnetic resonance images of the plan template 13 that are acquired by means of the radio-frequency coil 2. For the interventional procedure, the plan template 13 is then exchanged for the penetration template 3, wherein the interventional procedure takes place using the coordinate determination. As shown, the coordinate system 14 of the plan template 13 is designed analogous to the arrangement of the multiple guide devices 10 of the penetration template 3. Furthermore, the coordinate system 14 of the plan template 13 has magnetic resonance-visible markers at the coordinate points of the coordinate system 14. The coordinate system 14 is designed to be magnetic resonance-visible.

Although the invention has been illustrated and described in detail through the preferred exemplary embodiments, the invention is not limited by the disclosed examples, and other variations can be derived therefrom by those skilled in the art without departing from the scope of the invention. For example, the magnetic resonance marking element 9 can be designed differently than as is shown. The first and/or second mount 5, 7 and/or the first and/or second counterpart 6, 8 can also be designed differently than as shown, or cannot be used to fix the penetration template 3 in the opening 4.

We claim as our invention:

1. A coil system for an interventional magnetic resonance examination, comprising:
a penetration template;
a radio-frequency (RF) coil comprising an opening therein; and
said opening having a shape matched to a shape of said penetration template allowing said penetration template to be integrated into said opening.

2. A coil system as claimed in claim 1 wherein said penetration template is integrated with a positive fit into said opening.

3. A coil system as claimed in claim 1 wherein said RF coil comprises a mount situated at said opening, and wherein said penetration template comprises a counterpart that is matched to said mount, with said penetration template being fixed within said opening by interaction of said mount with said counterpart.

4. A coil system as claimed in claim 3 wherein said mount and said counterpart are configured to allow said penetration element to be removed from said opening after being fixed in said opening by said mount and said counterpart.

5. A coil system as claimed in claim 1 wherein said penetration template comprises a mount, and wherein said RF coil comprises a counterpart matched to said mount, and wherein said penetration template is affixed within said opening by interaction of said mount with said counterpart.

6. A coil system as claimed in claim 5 wherein said mount and said counterpart are configured to allow said penetration element to be removed from said opening after being fixed in said opening by said mount and said counterpart.

7. A coil system as claimed in claim 1 wherein said penetration template is comprised of magnetic resonance-compatible material.

8. A coil system as claimed in claim 1 comprising a magnetic resonance marking element situated at said opening or at said penetration template.

9. A coil system as claimed in claim 8 wherein said magnetic resonance marking element is formed by a channel in said RF coil or in said penetration template, said channel being filled with a magnetic resonance-visible fluid.

10. A coil system as claimed in claim 9 wherein said magnetic resonance-visible fluid is a magnetic resonance contrast agent.

11. A coil system as claimed in claim 1 comprising a plan template comprising a magnetic resonance-visible coordinate system thereon, said plan template being substitutable for said penetration template and being integrated into said opening of said RF coil.

12. A radio-frequency (RF) coil for a magnetic resonance apparatus, comprising:
a coil body comprising a coil; and
said coil body having an opening therein with a shape matched to a shape of a penetration template, allowing said penetration template to be integrated into said opening.

13. A radio-frequency coil as claimed in claim 12 comprising a mount situated at said opening of said coil body and matched to a counterpart of said penetration template, allowing said penetration template to be fixed in said opening by interaction of said mount with said counterpart.

14. A penetration template for an interventional magnetic resonance examination, comprising:
a template body comprising at least one guide device therein configured to guide an interventional device; and
a mount at a periphery of said template body matched to a counterpart of a radio-frequency coil, allowing said template body to be fixed within an opening of said radio-frequency coil by interaction of said mount with said counterpart.
